(19) United States
(12) Patent Application Publication
(10) Pub. No.: US 2013/0157320 A1
(43) Pub. Date: Jun. 20, 2013

(54) PROCESS FOR PRODUCING SULFUR-CONTAINING ALPHA-AMINO ACID COMPOUND

(71) Applicant: Hiroyuki Asako, Toyonaka-shi (JP)

(72) Inventor: Hiroyuki Asako, Toyonaka-shi (JP)

(73) Assignee: SUMITOMO CHEMICAL COMPANY, LIMITED, Tokyo (JP)

(21) Appl. No.: 13/703,287

(22) PCT Filed: Jun. 30, 2011
(86) PCT No.: PCT/JP2011/065528

§ 371 Date: Mar. 1, 2013

(30) Foreign Application Priority Data
Jun. 30, 2010 (JP) 2010-149338

(51) Int. Cl. C12P 13/04

(52) U.S. Cl. CPC C12P 13/04
USPC 435/106

(57) ABSTRACT

The present invention provides a novel process for producing a sulfur-containing α-amino acid compound such as methionine. A process for producing a sulfur-containing α-amino acid compound represented by the formula (2): wherein R¹ represents hydrogen, an alkyl group having 1 to 8 carbon atoms, or an aryl group having 6 to 20 carbon atoms; comprising: a first step of culturing a microorganism capable of converting a sulfur-containing amino alcohol compound represented by the formula (1): wherein R¹ is the same as defined above into the corresponding sulfur-containing α-amino acid compound in a culture medium containing a lower aliphatic alcohol to prepare a microbial cell of the microorganism; and a second step of reacting the sulfur-containing amino alcohol compound with the microbial cell of the microorganism obtained in the first step or a processed product of the microbial cell.

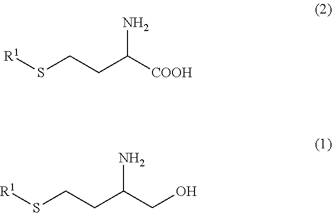

### TECHNICAL FIELD

The present invention relates to a process for producing a sulfur-containing α-amino acid compound.

### BACKGROUND ART

Hitherto, methionine, which is one of sulfur-containing α-amino acid compounds, has been used as an animal feed additive. In a process for producing methionine, acrolein and methyl mercaptan are reacted with each other to produce 3-methylthiopropionaldehyde, and then the 3-methylthiopropionaldehyde obtained is reacted with hydrogen cyanide, ammonia and carbon dioxide to produce 5-(2-methyl-mercaptoethyl)-hydantoin (that is, methionine hydantoin). The resultant product is hydrolyzed under an alkaline condition to give alkali metal methionate, followed by neutralization with an acid such as sulfuric acid or carbonic acid, to liberate methionine (see, for example, JP 55-102557 A).

### DISCLOSURE OF INVENTION

The above-mentioned process employs hydrogen cyanide as C1-building block and acrolein as C3-building block, which require careful safety control in handling and an equipment adopted to such control. Accordingly, there has been demand for a novel process for producing a sulfur-containing α-amino acid compound such as methionine.

An object of the present invention is to provide a novel process for producing a sulfur-containing α-amino acid compound such as methionine.

The present invention provides:

[1]A process for producing a sulfur-containing α-amino acid compound represented by the formula (2):

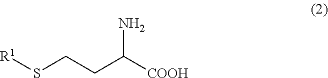

wherein R¹ represents hydrogen, an alkyl group having 1 to 8 carbon atoms, or an aryl group having 6 to 20 carbon atoms;
comprising

a first step of culturing a microorganism capable of converting a sulfur-containing amino alcohol compound represented by the formula (1):

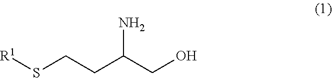

wherein R¹ is the same as defined above;
into a corresponding sulfur-containing α-amino acid compound (hereinafter, sometimes referred to as “the present microorganism”) in a culture medium containing a lower aliphatic alcohol to prepare a microbial cell of the microorganism (hereinafter, sometimes referred to as “the present catalytic cell”); and

a second step of reacting the sulfur-containing amino alcohol compound with the microbial cell of the microorganism obtained in the first step or a processed product of the microbial cell (hereinafter, the process of the item [1] sometimes referred to as “the process of the present invention”);

[2] The process according to the item [1] wherein the microorganism is capable of preferentially oxidizing the hydroxyl group of the sulfur-containing amino alcohol compound;
[3] The process according to the item [1] wherein the microorganism is one or more microorganisms selected from a group consisting of microorganisms of the genus Alcaligenes, microorganisms of the genus Bacillus, microorganisms of the genus Pseudomonas, microorganisms of the genus Rhodobacter and microorganisms of the genus Rhodococcus;
[4] The process according to the item [1] wherein the microorganism is one or more microorganisms selected from a group consisting of Alcaligenes denitrificans, Alcaligenes eutrophus, Alcaligenes faecalis, Alcaligenes sp., Alcaligenes xylosoxydans, Bacillus alvei, Bacillus badius, Bacillus brevis, Bacillus cereus, Bacillus coagulans, Bacillus firmus, Bacillus licheniformis, Bacillus moritai, Bacillus pumilus, Bacillus sphaericus, Bacillus subtilis, Bacillus validus, Pseudomonas denitrificans, Pseudomonas ficuserectae, Pseudomonas fragi, Pseudomonas mendocina, Pseudomonas oleovorans, Pseudomonas ovalis, Pseudomonas pseudoalcaligenes, Pseudomonas putida, Pseudomonas putrefaciens, Pseudomonas riboflavina, Pseudomonas straminea, Pseudomonas syringae, Pseudomonas tabaci, Pseudomonas taetrolens, Pseudomonas vesicularis, Rhodobacter sphaeroides, Rhodococcus erythropolis, Rhodococcus groberulus, Rhodococcus rhodochrous and Rhodococcus sp.;
[5] The process according to any one of the items [1] to [4] wherein R¹ of the sulfur-containing amino alcohol compound and the sulfur-containing α-amino acid compound is an alkyl group having 1 to 8 carbon atoms;
[6] The process according to any one of the items [1] to [4] wherein R¹ of the sulfur-containing amino alcohol compound and the sulfur-containing α-amino acid compound is a methyl group;
[7] The process according to any one of the items [1] to [6] wherein the lower aliphatic alcohol is a linear or a branched aliphatic alcohol having 1 to 5 carbon atoms; and
[8] The process according to any one of the items [1] to [6] wherein the lower aliphatic alcohol is at least one alcohol selected from the group consisting of methanol, ethanol, 1-propanol, 2-propanol, 1-butanol, tert-butanol, 2-methyl-1-propanol, 2,2-dimethyl-1-propanol, 1,2-butanediol and 1,3-butanediol.

The present invention is capable of providing a novel process for producing a sulfur-containing α-amino acid compound such as methionine.

### MODE FOR CARRYING OUT THE INVENTION

It will be understood that the inventions described herein is not limited to the particular methodologies, protocols, and reagents described herein and that they can be modified. It will be understood that the terms used herein are meant only to describe a particular embodiment of the present invention, and that such terms do not limit the scope of the present invention.

Unless otherwise noted, all of the technical terms and chemical terms used herein have the same meanings as those commonly understood by a person skilled in the technical field of the present invention. While the present invention may be carried out or examined by using methods or materials similar or equivalent to those described herein, some of the preferred methods, equipments, and materials are described in the following.

Hereinafter, the present invention is explained in more detail.

The process of the present invention comprises:

a first step of culturing a microorganism capable of converting a sulfur-containing amino alcohol compound represented by the formula (1):

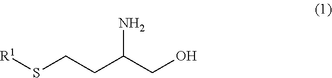

wherein R¹ represents hydrogen, an alkyl group having 1 to 8 carbon atoms, or an aryl group having 6 to 20 carbon atoms [hereinafter, sometimes referred to as “Compound (1)”], into the corresponding sulfur-containing α-amino acid compound, i.e. a compound represented by the formula (2):

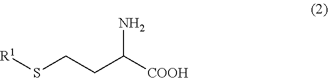

wherein R¹ is the same as defined above [hereinafter, sometimes referred to as “Compound (2)”] in a culture medium containing a lower aliphatic alcohol to prepare a microbial cell of the microorganism; and

a second step of reacting the sulfur-containing amino alcohol compound with the microbial cell of the microorganism obtained in the first step or a processed product of the microorganism cell.

Examples of “an alkyl group having 1 to 8 carbon atoms” represented by R¹ in Compound (1) and Compound (2) include a methyl group, an ethyl group, a propyl group, an isopropyl group, a butyl group, an isobutyl group, a t-butyl group, a pentyl group, a hexyl group, a heptyl group, and an octyl group. Examples of “an aryl group having 6 to carbon atoms” represented by R¹ include a phenyl group, a tolyl group, and a naphthyl group.

Preferred examples of R¹ include an alkyl group having 1 to 8 carbon atoms. More preferred examples of R¹ include a methyl group.

Examples of the “lower aliphatic alcohol” contained in the culture medium in the first step of the process of the present invention include a linear or a branched aliphatic alcohol having 1 to 5 carbon atoms. Specific examples of the “lower aliphatic alcohol” include methanol, ethanol, 1-propanol, 2-propanol, 1-butanol, tert-butanol, 2-methyl-1-propanol, 2,2-dimethyl-1-propanol, 1,2-butanediol, and 1,3-butanediol. Preferred examples of the “lower aliphatic alcohol” include 1-propanol, 1-butanol, 2,2-dimethyl-1-propanol, 1,2-butanediol, and 1,3-butanediol.

Any of these lower aliphatic alcohols may be mixed in the culture medium at an appropriate ratio.

A method for culturing the present microorganisms in a culture medium containing a lower aliphatic alcohol in the first step of the process of the present invention will be described later.

The microbial cell or the processed product of the microbial cell of a microorganism capable of preferentially oxidizing the hydroxyl group of the sulfur-containing amino alcohol compound, as a catalyst to be used in the process of the present invention, has an ability to convert Compound (1) into Compound (2). The activity of preferentially oxidizing the hydroxyl group can be improved by culturing the microorganism in a culture medium containing a lower aliphatic alcohol.

The term “preferentially oxidize” used herein means that the oxidation of a hydroxyl group proceeds preferentially to the oxidation of a sulfide group in the sulfur-containing amino alcohol compound.

Examples of the microorganism having the above ability (i.e. “the present microorganism”) include one or more microorganisms selected from a group consisting of microorganisms of the genus Alcaligenes, microorganisms of the genus Bacillus, microorganisms of the genus Pseudomonas, microorganisms of the genus Rhodobacter and microorganisms of the genus Rhodococcus.

Examples of the microorganism having the above ability (i.e. the present microorganism) also include one or more microorganisms selected from a group consisting of Alcaligenes denitrificans, Alcaligenes eutrophus, Alcaligenes faecalis, Alcaligenes sp., Alcaligenes xylosoxydans, Bacillus alvei, Bacillus badius, Bacillus brevis, Bacillus cereus, Bacillus coagulans, Bacillus firmus, Bacillus licheniformis, Bacillus moritai, Bacillus pumilus, Bacillus sphaericus, Bacillus subtilis, Bacillus validus, Pseudomonas denitrificans, Pseudomonas ficuserectae, Pseudomonas fragi, Pseudomonas mendocina, Pseudomonas oleovorans, Pseudomonas ovalis, Pseudomonas pseudoalcaligenes, Pseudomonas putida, Pseudomonas putrefaciens, Pseudomonas riboflavina, Pseudomonas straminea, Pseudomonas syringae, Pseudomonas tabaci, Pseudomonas taetrolens, Pseudomonas vesicularis, Rhodobacter sphaeroides, Rhodococcus erythropolis, Rhodococcus groberulus, Rhodococcus rhodochrous and Rhodococcus sp.

Preferred examples of the microorganism having the above ability include one or more microorganisms selected from a group consisting of Alcaligenes denitrificans JCM5490, Alcaligenes eutrophus ATCC43123, Alcaligenes faecalis IFO12669, Alcaligenes sp. IFO14130, Alcaligenes xylosoxydans IFO15125t, Alcaligenes xylosoxydans IFO15126t, Bacillus alvei IFO3343t, Bacillus badius ATCC14574t, Bacillus brevis JCM2503t, Bacillus cereus JCM2152t, Bacillus coagulans JCM2257t, Bacillus firmus JCM2512t, Bacillus licheniformis ATCC27811, Bacillus licheniformis IFO12197, Bacillus licheniformis IFO12200t, Bacillus moritai ATCC21282, Bacillus pumilus IFO12092t, Bacillus sphaericus IFO3341, Bacillus sphaericus IFO3526, Bacillus subtilis ATCC14593, Bacillus subtilis ATCC15841, Bacillus subtilis IFO3108, Bacillus subtilis IFO3132, Bacillus subtilis IFO3026, Bacillus subtilis IFO3037, Bacillus subtilis IFO3108, Bacillus subtilis IFO3134, Bacillus validus IFO013635, Pseudomonas denitrificans IAM1426, Pseudomonas denitrificans IAM1923, Pseudomonas ficuserectae JCM2400t, Pseudomonas fragi IAM12402, Pseudomonas fragi IFO3458t, Pseudomonas mendocina IFO14162, Pseudomonas oleovorans IFO13583t, Pseudomonas ovalis IFO12688, Pseudomonas pseudoalcaligenes JCM5968t, Pseudomonas putida IFO12996, Pseudomonas putida IFO014164t, Pseudomonas putida IFO3738, Pseudomonas putida IFO12653, Pseudomonas putrefaciens IFO3910, Pseudomonas riboflavina IFO13584t, Pseudomonas straminea JCM2783t, Pseudomonas syringae IFO14055, Pseudomonas tabaci IFO3508, Pseudomonas taetrolens IFO3460, Pseudomonas vesicularis JCM1477t, Rhodobacter sphaeroides ATCC17023, Rhodococcus erythropolis IFO12320, Rhodococcus groberulus ATCC15076, Rhodococcus rhodochrous ATCC15076, Rhodococcus rhodochrous ATCC15610, Rhodococcus rhodochrous ATCC19067, Rhodococcus rhodochrous ATCC19149, Rhodococcus rhodochrous ATCC19150, Rhodococcus rhodochrous ATCC21197, Rhodococcus rhodochrous ATCC21199, Rhodococcus rhodochrous JCM3202t, Rhodococcus sp. ATCC19070, Rhodococcus sp. ATCC19071 and Rhodococcus sp. ATCC19148.

More preferred examples of the microorganism having the above ability include one or more microorganisms selected from a group consisting of Rhodococcus erythropolis IFO12320, Rhodococcus groberulus ATCC15076, Rhodococcus rhodochrous ATCC15076, Rhodococcus rhodochrous ATCC15610, Rhodococcus rhodochrous ATCC19067, Rhodococcus rhodochrous ATCC19149, Rhodococcus rhodochrous ATCC19150, Rhodococcus rhodochrous ATCC21197, Rhodococcus rhodochrous ATCC21199, Rhodococcus rhodochrous JCM3202t, Rhodococcus sp. ATCC19070, Rhodococcus sp. ATCC19071 and Rhodococcus sp. ATCC19148.

These microorganisms may be either isolated from natural sources, or easily gotten by purchasing from a culture collection.

Examples of culture collection from which the microorganisms can be purchased include the following culture collections.

### 1. IFO (Institute of Fermentation Osaka) Culture Collection

At present, the culture collection is transferred to National Institute of Technology and Evaluation Biological Resource Center (NBRC). Microorganisms can be purchased by filing an application to NBRC, which can be done by, for example, accessing the website of NBRC (http://www.nbrc.nite.go.jp/NBRC2/NBRCDispSearchServlet?lan g=jp).

### 2. ATCC (American Type Culture Collection)

Microorganisms can be purchased through Summit Pharmaceuticals International Corporation, ATCC Industry Division by, for example, accessing its website (http://www.summitpharma.co.jp/japanese/service/s_ATCC.html). Alternatively, microorganisms can be purchased directly from ATCC.

### 3. JCM (Japan Collection of Microorganisms)

At present, the culture collection is transferred to National Institute of Physical and Chemical Research Biological Resource Center (RIKEN BRC), Microbe Division. Microorganisms can be purchased by filing an application to RIKEN BRC, which can be done by, for example, accessing a site for culture collection in the website of RIKEN (http://www.jcm.riken.go.jp/JCM/aboutJCM_J.shtml).

### 4. IAM Culture Collection

At present, among the IAM Culture Collection, bacteria, yeasts, and filamentous fungi are transferred to National Institute of Physical and Chemical Research Biological Resource Center, Microbe Division (JCM), and microalgae are transferred to Microbial Culture Collection in National Institute for Environmental Studies (NIES). Microorganisms can be purchased by filing an application to JCM or NIES, which can be done by, for example, accessing a site for the culture collections in the website of JCM (http://www.jcm.riken.go.jp/JCM/aboutJCM_J.shtml) or in the website of NIES (http://mcc.nies.go.jp/aboutOnlineOrder.do).

The microbial cell or the processed products of the microbial cell of a microorganism capable of preferentially oxidize the hydroxyl group of the sulfur-containing amino alcohol compound, as a catalyst to be used in the process of the present invention, may also be obtained and prepared by screening a microorganism which is capable of converting Compound (1) into Compound (2) and which is capable of improving its activity to preferentially oxidize the hydroxyl group when it is cultured in a culture medium containing a lower aliphatic alcohol.

Described as follows is a procedure for screening a microorganism capable of converting Compound (1) into Compound (2).

Specifically, for example, in a test tube is placed 5 ml of sterilized culture medium, and thereto is inoculated with a microorganism obtained by purchasing from a culture collection or a microorganism isolated from soil. The resultant is incubated with shaking at 30° C. under an aerobic condition. After the completion of the incubation, the microbial cells are collected by centrifugation to obtain viable cells. In a screw-top test tube is placed 2 ml of 0.1 M Tris-glycine buffer (pH 10), and thereto are added the above-prepared viable cells, and the mixture is suspended. To the suspension is added 2 mg of methioninol, and the resultant mixture is shaken at 30° C. for 3 to 7 days.

After the completion of the reaction, 1 ml of the reaction solution is sampled. The cells are removed from the solution sample, and the amount of the produced methionine is analyzed by liquid chromatography.

Thus, a microorganism capable of converting Compound (1) into Compound (2) may be screened.

Described as follows is a procedure for screening a microorganism capable of improving its activity to preferentially oxidize the hydroxyl group when it is cultured in a culture medium containing a lower aliphatic alcohol.

Specifically, for example, in a test tube is placed 5 ml of sterilized culture medium containing a lower aliphatic alcohol, which is prepared by adding a lower aliphatic alcohol (5 g), polypeptone (5 g), yeast extract (3 g), meat extract (3 g), ammonium sulfate (0.2 g), potassium dihydrogen phosphate (1 g) and magnesium sulfate heptahydrate (0.5 g) to 1 L of water and then adjusting the pH to 7.0, and thereto is inoculated with a microorganism obtained by purchasing from a culture collection or a microorganism isolated from soils. The resultant is incubated with shaking at 30° C. under an aerobic condition. After the completion of the incubation, the microbial cells are collected by centrifugation to obtain viable cells. In a screw-top test tube is placed 2 ml of 0.1 M Tris-glycine buffer (pH 10), and thereto is added the above-prepared viable cells, and the mixture is suspended. To the suspension is added 2 mg of methioninol, and the resultant mixture is shaken at 30° C. for 3 to 7 days.

After the completion of the reaction, 1 ml of the reaction solution is sampled. The cells are removed from the solution sample, and the amount of the produced methionine is analyzed by liquid chromatography.

Meanwhile, the amount of the produced methionine is also analyzed in a reaction solution obtained by conducting the same procedure as the above except that the microorganism has been cultured in a culture medium not containing a lower aliphatic alcohol and, the “amount of the produced methionine” obtained is compared with the above “amount of the produced methionine”.

Thus, screened may be a microorganism capable of improving the activity to preferentially oxidize the hydroxyl group when it is cultured in a culture medium containing a lower aliphatic alcohol.

Described as follows is a method for the preparation of the present microorganisms.

The present microorganism may be cultured in a culture medium for culturing various microorganisms, the culture medium appropriately containing a carbon source, a nitrogen source, an organic salt, an inorganic salt, and so on.

Examples of the carbon source include sugars such as glucose, dextrin and sucrose; sugar alcohols such as glycerol; organic acids such as fumaric acid, citric acid and pyruvic acid; animal oils; vegetable oils; and molasses. These carbon sources are added to the culture medium in an amount of usually about 0.1% (w/v) to 30% (w/v) of the culture.

Examples of the nitrogen source include natural organic nitrogen sources such as meat extract, peptone, yeast extract, malt extract, soy flour, Corn Steep Liquor, cottonseed flour, dried yeast and casamino acids; amino acids; sodium salts of inorganic acids such as sodium nitrate; ammonium salts of inorganic acids such as ammonium chloride, ammonium sulfate and ammonium phosphate; ammonium salts of organic acids such as ammonium fumarate and ammonium citrate; and urea. Among these nitrogen sources, ammonium salts of organic acids, natural organic nitrogen sources, and amino acids and others may also be used as carbon sources in many cases. The above nitrogen sources are added to the culture medium in an amount of usually about 0.1% (w/v) to 30% (w/v) of the culture.

Examples of the organic salt and inorganic salt include chloride, sulfate, acetate, carbonate, and phosphate of potassium, sodium, magnesium, iron, manganese, cobalt, and zinc. Specific examples thereof include sodium chloride, potassium chloride, magnesium sulfate, ferrous sulfate, manganese sulfate, cobalt chloride, zinc sulfate, copper sulfate, sodium acetate, calcium carbonate, potassium hydrogen phosphate and dipotassium hydrogen phosphate. These organic salts and/or inorganic salts are added to the culture medium in an amount of usually about 0.0001% (w/v) to 5% (w/v) of the culture.

Examples of the culture method include solid culture and liquid culture (e.g. a test tube culture, a flask culture, or a jar fermenter culture).

Culture temperature and pH of the culture are not particularly limited as long as the present microorganisms are able to grow in the range thereof. For example, the culture temperature may be in a range of about 15° C. to about 45° C., and the pH of the culture may be in a range of about 4 to about 8. The culture time may be appropriately selected depending on the culture conditions, and is usually about 1 day to about 7 days.

The present microorganism obtained in this manner is cultured in a culture medium containing a lower aliphatic alcohol in the first step of the present invention to provide the microbial cell of the microorganism (i.e. the present catalytic cell). The microbial cell of the microorganism obtained in the first step (i.e. the present catalytic cell) or a processed product of the microbial cell is used as “a catalyst of the process of the present invention” in the second step of the process of the present invention.

Described as follows is a method for culturing the present microorganism in a culture medium containing a lower aliphatic alcohol in the first step of the process of the present invention.

The present microorganism may be cultured in a culture medium for culturing various microorganisms, the culture medium appropriately containing a carbon source, a nitrogen source, an organic salt, an inorganic salt, and so on.

As the carbon source contained in the culture medium, a lower aliphatic alcohol alone may be used or a mixture of sugars, hydrocarbons, organic acids, sugar alcohols and others may be used.

As previously mentioned, examples of the “lower aliphatic alcohol” contained in the culture medium used in the first step of the process of the present invention include a linear or a branched aliphatic alcohol having 1 to 5 carbon atoms. Specific examples of the “lower aliphatic alcohol” include methanol, ethanol, 1-propanol, 2-propanol, 1-butanol, tert-butanol, 2-methyl-1-propanol, 2,2-dimethyl-1-propanol, 1,2-butanediol, and 1,3-butanediol. Preferred examples of the “lower aliphatic alcohol” include 1-propanol, 1-butanol, 2,2-dimethyl-1-propanol, 1,2-butanediol, and 1,3-butanediol.

Any of these lower aliphatic alcohols may be mixed in the culture medium at an appropriate ratio.

As previously mentioned, the carbon source may be a lower aliphatic alcohol. These carbon sources are added to the culture medium in an amount of usually about 0.1% (w/v) to 30% (w/v) of the culture.

Examples of the nitrogen source include natural organic nitrogen sources such as meat extract, peptone, yeast extract, malt extract, soy flour, Corn Steep Liquor, cottonseed flour, dried yeast, and casamino acids; amino acids; sodium salts of inorganic acids such as sodium nitrate; ammonium salts of inorganic acids such as ammonium chloride, ammonium sulfate and ammonium phosphate; ammonium salts of organic acids such as ammonium fumarate and ammonium citrate; and urea. Among these nitrogen sources, ammonium salts of organic acids, natural organic nitrogen sources, and amino acids and others may also be used as carbon sources in many cases. The above nitrogen sources are added to the culture medium in an amount of usually about 0.1% (w/v) to 30% (w/v) of the culture.

Examples of the organic salt and inorganic salt include chloride, sulfate, acetate, carbonate and phosphate of potassium, sodium, magnesium, iron, manganese, cobalt, and zinc. Specific examples thereof include sodium chloride, potassium chloride, magnesium sulfate, ferrous sulfate, manganese sulfate, cobalt chloride, zinc sulfate, copper sulfate, sodium acetate, calcium carbonate, potassium hydrogen phosphate and dipotassium hydrogen phosphate. These organic salts and/or inorganic salts are added to the culture medium in an amount of usually about 0.0001% (w/v) to 5% (w/v) of the culture.

Examples of the culture method include solid culture and liquid culture (e.g. a test tube culture, a flask culture, or a jar fermenter culture).

Culture temperature and pH of the culture are not particularly limited as long as the present microorganisms are able to grow in the range thereof. For example, the culture temperature may be in a range of about 15° C. to about 45° C., and the pH of the culture may be in a range of about 4 to about 8. The culture time may be appropriately selected depending on the culture conditions, and is usually about 1 day to about 7 days.

The present catalytic cell can be directly used as a catalyst for the process of the present invention. Among methods for using the present catalytic cell, examples of a method for directly using the present catalytic cell include:

(1) a method for directly using a culture, and

(2) a method for using microbial cells collected by centrifuging a culture (wet microbial cells washed as needed with buffer or water).

The processed products of the present catalytic cell may also be used as a catalyst for the process of the present invention. Examples of the processed product include microbial cells obtained by culturing followed by treating with an organic solvent (e.g. acetone and ethanol), lyophilizing, or treating with alkali; physically or enzymatically disrupted microbial cells; and crude enzymes separated or extracted from the these microbial cells. Furthermore, examples of the processed products include those immobilized by a known method after the above-mentioned treatments.

Specific embodiments include the present catalytic cell and the processed products thereof (e.g. cell-free extracts, partially purified proteins, purified proteins and immobilized materials thereof). Examples of the processed products include lyophilized microorganisms, organic solvent-treated microorganisms, dried microorganisms, disrupted microorganisms, autolysates of microorganisms, sonicated microorganisms, extracts of microorganisms, and alkali-treated microorganisms. Examples of a method of obtaining the immobilized materials include carrier binding methods (e.g. a method of adsorbing proteins and others onto inorganic carriers such as silica gel and ceramics, cellulose, or ion-exchanged resin) and encapsulating methods [e.g. a method of trapping proteins and others in a network structure of macromolecules such as polyacrylamide, sulfur-containing polysaccharide gel (e.g. carrageenan gel), alginate gel, and agar gel].

In the event that the present catalytic cell is used in the industrial production process, the product of killed microorganisms might be preferred to unprocessed microorganisms from the point of view of limitation of manufacturing equipments or other factors. Examples of a method for killing the microorganism include physical sterilization (e.g. heating, drying, freezing, irradiation, sonication, filtration, and electric sterilization) and sterilization with chemical agents (e.g. alkalis, acids, halogens, oxidizing agents, sulfur, boron, arsenic, metals, alcohols, phenols, amines, sulfides, ethers, aldehydes, ketones, cyan, and antibiotics). Among these killing methods, generally, it is preferable to select a method which can lower the amount of residues or contaminants in the reaction system and can minimize inactivation of the above-described ability of the present microorganism to preferentially oxidize the hydroxyl group of the sulfur-containing amino alcohol compound.

The second step of the process of the present invention is usually carried out in the presence of water. The water used in this case may be in the form of a buffer. Examples of buffering agents used in the buffer include alkali metal salts of phosphoric acid such as sodium phosphate and potassium phosphate, and alkali metal salts of acetic acid such as sodium acetate and potassium acetate. Examples of alkaline buffer include Tris-HCl buffer, Tris-citrate buffer, and Tris-glycine buffer.

The second step of the process of the present invention may also be carried out by additionally using a hydrophobic organic solvent, i.e. in the presence of water and the hydrophobic organic solvent. Examples of the hydrophobic organic solvent used in this case include esters such as ethyl formate, ethyl acetate, propyl acetate, butyl acetate, ethyl propionate and butyl propionate, alcohols such as n-butyl alcohol, n-amyl alcohol and n-octyl alcohol, aromatic hydrocarbons such as benzene, toluene and xylene, ethers such as diethylether, diisopropylether and methyl-t-butylether, halogenated hydrocarbons such as chloroform and 1,2-dichloroethane, and mixtures thereof.

The second step of the process of the present invention may also be carried out by additionally using a hydrophilic organic solvent, i.e. in the presence of water and an aqueous medium. Examples of the hydrophilic organic solvent used in this case include alcohols such as methanol and ethanol, ketones such as acetone, ethers such as dimethoxyethane, tetrahydrofuran and dioxane, dimethylsulfoxide, and mixtures thereof.

While the second step of the process of the present invention is usually carried out in a range of pH of aqueous layer of 3 to 11, the pH may be appropriately changed in such a range that the reaction proceeds. It is preferable that the process of the present invention be carried out in the alkaline range, and it is more preferable that the process be carried out in a range of pH of aqueous layer of 8 to 10.

While the second step of the process of the present invention is usually carried out in a range of about 0° C. to about 60° C., the temperature may be appropriately changed in such a range that the reaction proceeds.

The second step of the process of the present invention is usually carried out in a range of for about 0.5 hours to about 10 days. After the completion of adding the sulfur-containing amino alcohol compound represented by the formula (1) [i.e. Compound (1)], which is the starting compound, the endpoint of the reaction can be checked, for example, by measuring the amount of the sulfur-containing amino alcohol compound of the formula (1) in the reaction solution by liquid chromatography or gas chromatography and the others.

The concentration of the sulfur-containing amino alcohol compound represented by the formula (1) [i.e. Compound (1)], which is the starting compound used in the second step of the process of the present invention is usually 50% (w/v) or less and the sulfur-containing amino alcohol compound of the formula (1) [i.e. Compound (1)] may be continuously or successively added to a reaction system in order to maintain the concentration of the sulfur-containing amino alcohol compound of the formula (1) in the reaction system nearly constant.

During the second step of the process of the present invention, for example, a sugar such as glucose, sucrose or fructose, or a surfactant such as Triton X-100 (registered trade mark) or Tween 60 (registered trade mark)) may be added to the reaction system if necessary.

The recover of the sulfur-containing α-amino acid compound represented by the formula (2) from the reaction solution may be carried out by any methods known in the art.

Example of the method include purification by performing post-treatment of the reaction solution such as organic solvent extraction, concentration, ion exchange method and crystallization, if necessary in combination with column chromatography, and distillation and others.

The sulfur-containing amino acid compound represented by the formula (2) prepared in the second step of the process of the present invention may be in the form of a salt.

### EXAMPLES

Hereinafter, the present invention is explained in more detail with some examples.

### Example 1

### Production of the Sulfur-Containing α-Amino Acid Compound From the Sulfur-Containing Amino Alcohol Compound According To the Process of the Present Invention

In a test tube was placed 5 ml of sterilized culture medium, which was prepared by adding each of lower aliphatic alcohols shown in Tables 2 to 4 (5 g), polypeptone (5 g), yeast extract (3 g), meat extract (3 g), ammonium sulfate (0.2 g), potassium dihydrogen phosphate (1 g) and magnesium sulfate heptahydrate (0.5 g) to 1 L of water and adjusting the pH to 7.0, and thereto was inoculated with each cells of Rhodococcus rhodochrous ATCC19149 (Table 1), Rhodococcus rhodochrous ATCC19150 (Table 2), Rhodococcus sp. ATCC19070 (Table 3), or Rhodococcus sp. ATCC19148 (Table 4). The resultant was incubated with shaking at 30° C. under an aerobic condition. After the completion of the incubation, the microbial cells were collected by centrifugation to obtain viable cells. In a screw-top test tube was placed 2 ml of 0.1 M Tris-glycine buffer (pH 10), and thereto was added the above-prepared viable cells, and the mixture was suspended. To the suspension was added 2 mg of starting materials (i.e. methioninol), and the resultant mixture was shaken at 30° C. for 7 to 10 days.

After the completion of the reaction, 0.5 ml of the reaction solution was sampled. The cells were removed from the solution sample, and the amount of the produced methionine was analyzed by liquid chromatography. The results are shown in Tables 1 to 4.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min.
Column temperature: 40° C.

### Detection: 220 nm

TABLE 1

| The added lower | Methionine |
|---|---|
| aliphatic alcohol | yield (%) |
| 1-Propanol | 51.6 |
| 1-Butanol | 32.0 |
| 1,2-Butanediol | 44.3 |
| 2,2-Dimethyl-1-propanol | 76.4 |
| 1,3-Butanediol | 54.0 |
| No addition | 31.6 |

TABLE 2

| The added lower | Methionine |
|---|---|
| aliphatic alcohol | yield (%) |
| 1-Propanol | 38.8 |
| 1-Butanol | 32.7 |
| 1,2-Butanediol | 46.6 |
| 2,2-Dimethyl-1-propanol | 52.8 |
| 1,3-Butanediol | 51.0 |
| No addition | 30.0 |

TABLE 3

| The added lower | Methionine |
|---|---|
| aliphatic alcohol | yield (%) |
| 1-Propanol | 48.4 |
| 1-Butanol | 55.7 |
| 1,2-Butanediol | 49.0 |
| 2,2-Dimethyl-1-propanol | 77.2 |
| 1,3-Butanediol | 61.3 |
| No addition | 35.3 |

TABLE 4

| The added lower | Methionine |
|---|---|
| aliphatic alcohol | yield (%) |
| 1-Propanol | 61.9 |
| 1-Butanol | 56.3 |
| 1,2-Butanediol | 40.3 |
| 2,2-Dimethyl-1-propanol | 54.3 |
| 1,3-Butanediol | 35.7 |
| No addition | 32.1 |

### Example 2

### Production of the Sulfur-Containing α-Amino Acid Compound From the Sulfur-Containing Amino Alcohol Compound According To the Process of the Present Invention

In a test tube was placed 5 ml of sterilized culture medium, which was prepared by adding each of lower aliphatic alcohols shown in Table 5 (5 g), polypeptone (5 g), yeast extract (3 g), meat extract (3 g), ammonium sulfate (0.2 g), potassium dihydrogen phosphate (1 g) and magnesium sulfate heptahydrate (0.5 g) to 1 L of water and adjusting the pH to 7.0, and thereto was inoculated with Rhodococcus groberulus ATCC15076. The resultant was incubated with shaking at 30° C. under an aerobic condition. After the completion of the incubation, the microbial cells were collected by centrifugation to obtain viable cells. In a screw-top test tube was placed 2 ml of 0.1 M Tris-glycine buffer (pH 10), and thereto was added the above-prepared viable cells, and the mixture was suspended. To the suspension was added 2 mg of starting materials (i.e. methioninol), and the resultant mixture was shaken at 30° C. for 4 days.

After the completion of the reaction, 0.5 ml of the reaction solution was sampled. The cells were removed from the sampling solution, and the amount of the produced methionine was analyzed by liquid chromatography. The results are shown in Table 5.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min.
Column temperature: 40° C.

### Detection: 220 nm

TABLE 5

| The added lower | Methionine |
|---|---|
| aliphatic alcohol | yield (%) |
| Methanol | 29.3 |
| Ethanol | 30.4 |
| 1-Propanol | 48.1 |
| 2-Propanol | 36.8 |
| 1-Butanol | 31.5 |
| Tert-butanol | 52.5 |
| 2-Methyl-1-propanol | 48.1 |
| 2,2-Dimethyl-1-propanol | 56.2 |
| 1,2-Butanediol | 35.9 |
| 1,3-Butanediol | 31.3 |
| No addition | 23.5 |

### Reference Example 1

### Production of the Sulfur-Containing α-Amino Acid Compound From the Sulfur-Containing Amino Alcohol Compound by Using The Present Microorganism

In a test tube was placed 5 ml of sterilized culture medium, which was prepared by adding polypeptone (5 g), yeast extract (3 g), meat extract (3 g), ammonium sulfate (0.2 g), potassium dihydrogen phosphate (1 g) and magnesium sulfate heptahydrate (0.5 g) to 1 L of water and adjusting the pH to 7.0, and thereto was inoculated with each cells of the microorganisms shown in Table 6. The resultant was incubated with shaking at 30° C. under an aerobic condition. After the completion of the incubation, the microbial cells were collected by centrifugation to obtain viable cells. In a screw-top test tube was placed 2 ml of 0.1 M Tris-glycine buffer (pH 10), and thereto was added the above-prepared viable cells, and the mixture was suspended. To the suspension was added 2 mg of methioninol, and the resultant mixture was shaken at 30° C. for 3 to 7 days.

After the completion of the reaction, 1 ml of the reaction solution was sampled. The cells were removed from the solution sample, and the amount of the produced methionine was analyzed by liquid chromatography. The results are shown in Table 6.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min.

Column temperature: 40° C.

Detection: 220 nm

TABLE 6

| | Methionine |
|---|---|
| Name of strain | yield (%) |
| Alcaligenes denitrificans JCM 5490 | 15.9 |
| Alcaligenes eutrophus ATCC 43123 | 18.2 |
| Alcaligenes faecalis IFO 12669 | 13.7 |
| Alcaligenes sp. IFO 14130 | 16.5 |
| Alcaligenes xylosoxydans IFO15125t denitrificans | 16.8 |
| Alcaligenes xylosoxydans IFO15126t xylosoxydans | 15.8 |
| Bacillus alvei IFO 3343t | 14.3 |
| Bacillus badius ATCC 14574t | 15.6 |
| Bacillus brevis JCM 2503t | 15.0 |
| Bacillus cereus JCM 2152t | 17.0 |
| Bacillus coagulans JCM 2257t | 10.7 |
| Bacillus firmus JCM 2512t | 18.3 |
| Bacillus licheniformis ATCC 27811 | 16.6 |
| Bacillus licheniformis IFO 12197 | 17.0 |
| Bacillus licheniformis IFO 12200t | 10.5 |
| Bacillus moritai ATCC 21282 | 16.7 |
| Bacillus pumilus IFO 12092t | 16.2 |
| Bacillus sphaericus IFO 3341 | 17.2 |
| Bacillus sphaericus IFO 3526 | 18.3 |
| Bacillus subtilis ATCC 14593 | 13.9 |
| Bacillus subtilis ATCC15841 | 12.1 |
| Bacillus subtilis IFO 03108 | 14.0 |
| Bacillus subtilis IFO 03134 | 15.0 |
| Bacillus subtilis IFO 3026 | 18.9 |
| Bacillus subtilis IFO 3037 | 14.3 |
| Bacillus subtilis IFO 3108 | 13.3 |
| Bacillus subtilis IFO 3134 | 12.6 |
| Bacillus validus IFO 13635 | 14.4 |
| Pseudomonas denitrificans IAM 1426 | 17.2 |
| Pseudomonas denitrificans IAM 1923 | 36.5 |
| Pseudomonas ficuserectae JCM 2400t | 19.3 |
| Pseudomonas fragi IAM 12402 | 16.0 |
| Pseudomonas fragi IFO 3458t | 34.3 |
| Pseudomonas mendocina IFO 14162 | 26.8 |
| Pseudomonas oleovorans IFO 13583t | 19.6 |
| Pseudomonas ovalis IFO 12688 | 12.7 |
| Pseudomonas pseudoalcaligenes JCM 5968t | 15.2 |
| Pseudomonas putida IFO 12996 | 14.3 |
| Pseudomonas putida IFO 14164t | 14.4 |
| Pseudomonas putida IFO 3738 | 29.2 |
| Pseudomonas putida IFO12653 | 12.8 |
| Pseudomonas putrefaciens IFO 3910 | 16.4 |
| Pseudomonas riboflavina IFO 13584t | 11.9 |
| Pseudomonas straminea JCM 2783t | 23.7 |
| Pseudomonas syringae subsp. syringae IFO14055 | 23.7 |
| Pseudomonas tabaci IFO 3508 | 12.5 |
| Pseudomonas taetrolens IFO 3460 | 18.1 |
| Pseudomonas vesicularis JCM 1477t | 15.3 |
| Rhodobacter sphaeroides ATCC 17023 | 15.7 |
| Rhodococcus erythropolis IFO 12320 | 12.4 |
| Rhodococcus globerulus ATCC 15076 | 28.9 |
| Rhodococcus rhodochrous ATCC 15610 | 22.3 |
| Rhodococcus rhodochrous ATCC 19067 | 25.4 |
| Rhodococcus rhodochrous ATCC 19149 | 22.5 |
| Rhodococcus rhodochrous ATCC 19150 | 24.9 |
| Rhodococcus rhodochrous ATCC 21197 | 26.5 |
| Rhodococcus rhodochrous ATCC 21199 | 27.2 |
| Rhodococcus rhodochrous JCM 3202t | 38.5 |
| Rhodococcus sp ATCC 19070 | 22.5 |
| Rhodococcus sp ATCC 19071 | 35.8 |
| Rhodococcus sp ATCC 19148 | 24.8 |

### Reference Example 2

### Screening Microorganisms Capable of Converting the Sulfur-Containing Amino Alcohol Compound into a Corresponding Sulfur-Containing α-Amino Acid Compound

In a test tube is placed 5 ml of sterilized culture medium, which is prepared by adding polypeptone (5 g), yeast extract (3 g), meat extract (3 g), ammonium sulfate (0.2 g), potassium dihydrogen phosphate (1 g) and magnesium sulfate heptahydrate (0.5 g) to 1 L of water and then adjusting the pH to 7.0, and thereto is inoculated with a microorganism obtained by purchasing from a culture collection or a microorganism isolated from soils. The resultant is incubated with shaking at 30° C. under an aerobic condition. After the completion of the incubation, the microbial cells are collected by centrifugation to obtain viable cells. In a screw-top test tube is placed 2 ml of 0.1 M Tris-glycine buffer (pH 10), and thereto is added the above-prepared viable cells, and the mixture is suspended. To the suspension is added 2 mg of methioninol, and the resultant mixture is shaken at 30° C. for 3 to 7 days.

After the completion of the reaction, 1 ml of the reaction solution is sampled. The cells are removed from the solution sample, and the amount of the produced methionine is analyzed by liquid chromatography.

Thus, microorganisms capable of converting the sulfur-containing amino alcohol compound into the corresponding sulfur-containing α-amino acid compound are screened.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### INDUSTRIAL APPLICABILITY

The present invention can provide a novel process for producing a sulfur-containing α-amino acid compound such as methionine.

1. A process for producing a sulfur-containing α-amino acid compound represented by the formula (2): 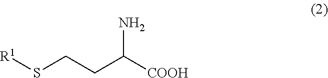
wherein R¹ represents hydrogen, an alkyl group having 1 to 8 carbon atoms, or an aryl group having 6 to 20 carbon atoms;
comprising:
a first step of culturing a microorganism capable of converting a sulfur-containing amino alcohol compound represented by the formula (1): 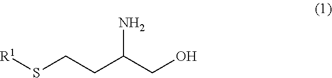
wherein R¹ is the same as defined above;
into the corresponding sulfur-containing α-amino acid compound in a culture medium containing a lower aliphatic alcohol to prepare a microbial cell of the microorganism; and
a second step of reacting the sulfur-containing amino alcohol compound with the microbial cell of the microorganism obtained in the first step or a processed product of the microbial cell.

2. The process according to claim 1 wherein the microorganism is capable of preferentially oxidizing the hydroxyl group of the sulfur-containing amino alcohol compound.

3. The process according to claim 1 wherein the microorganism is one or more microorganisms selected from a group consisting of microorganisms of the genus Alcaligenes, microorganisms of the genus Bacillus, microorganisms of the genus Pseudomonas, microorganisms of the genus Rhodobacter and microorganisms of the genus Rhodococcus.

4. The process according to claim 1 wherein the microorganism is one or more microorganisms selected from a group consisting of Alcaligenes denitrificans, Alcaligenes eutrophus, Alcaligenes faecalis, Alcaligenes sp., Alcaligenes xylosoxydans, Bacillus alvei, Bacillus badius, Bacillus brevis, Bacillus cereus, Bacillus coagulans, Bacillus firmus, Bacillus licheniformis, Bacillus moritai, Bacillus pumilus, Bacillus sphaericus, Bacillus subtilis, Bacillus validus, Pseudomonas denitrificans, Pseudomonas ficuserectae, Pseudomonas fragi, Pseudomonas mendocina, Pseudomonas oleovorans, Pseudomonas ovalis, Pseudomonas pseudoalcaligenes, Pseudomonas putida, Pseudomonas putrefaciens, Pseudomonas riboflavina, Pseudomonas straminea, Pseudomonas syringae, Pseudomonas tabaci, Pseudomonas taetrolens, Pseudomonas vesicularis, Rhodobacter sphaeroides, Rhodococcus erythropolis, Rhodococcus groberulus, Rhodococcus rhodochrous and Rhodococcus sp.

5. The process according to any one of claims 1 to 4 wherein R¹ of the sulfur-containing amino alcohol compound and the sulfur-containing α-amino acid compound is an alkyl group having 1 to 8 carbon atoms.

6. The process according to any one of claims 1 to 4 wherein R¹ of the sulfur-containing amino alcohol compound and the sulfur-containing α-amino acid compound is a methyl group.

7. The process according to claim 1 wherein the lower aliphatic alcohol is a linear or a branched aliphatic alcohol having 1 to 5 carbon atoms.

8. The process according to claim 1 wherein the lower aliphatic alcohol is at least one alcohol selected from the group consisting of methanol, ethanol, 1-propanol, 2-propanol, 1-butanol, tert-butanol, 2-methyl-1-propanol, 2,2-dimethyl-1-propanol, 1,2-butanediol and 1,3-butanediol.
